(19) United States
(12) Patent Application Publication
(10) Pub. No.: US 2007/0265300 A1
(43) Pub. Date: Nov. 15, 2007

(54) Pyridine Derivatives of Alkyl Oxindoles as 5-Ht7 Receptor Active Agents

(75) Inventors: Balazs Volk, Budapest (HU); Jozsef Barkoczy, Budapest (HU); Gyula Simig, Budapest (HU); Tibor Mezei, Budapest (HU); Rita Kapillerne Dezsofi, Budapest (HU); Istvan Gacsalyi, Budapest (HU); Katalin Pallagi, Budapest (HU); Gabor Gigler, Budapest (HU); Gyorgy Levay, Badakeszi (HU); Krisztina Moricz, Budapest (HU); Csilla Leveleki, Budapest (HU); Nora Sziray, Budapest (HU); Gabor Szenasi, Urom (HU); Andras Egyed, Budapest (HU); Laszlo Gabor Harsing, Budapest (HU)

Correspondence Address: BIRCH STEWART KOLASCH & BIRCH, PO BOX 747, FALLS CHURCH, VA, 22040-0747, US

(73) Assignee: Egis gyogyszergyar rt., Budapest (HU)

(21) Appl. No.: 11/596,472

(22) PCT Filed: May 10, 2005
(86) PCT No.: PCT/HU05/00047

§ 371 Date: Mar. 22, 2007

(30) Foreign Application Priority Data
May 11, 2004 (HU) P0400956
May 5, 2005 (HU) P0500462

(51) Int. Cl. A61K 31/404; A61K 31/44; A61K 31/47; A61P 25/00; C07D 209/34; C07D 217/00; C07D 401/12

(52) U.S. Cl. 514/307; 514/339; 514/418; 546/148; 546/277.7; 548/486

(57) ABSTRACT

The present invention is concerned with new 3,3-disubstituted indol-2-one derivatives of the general Formula (I). Compounds according to the invention are useful for the prophylaxis or treatment of the disorders of the central nervous system.

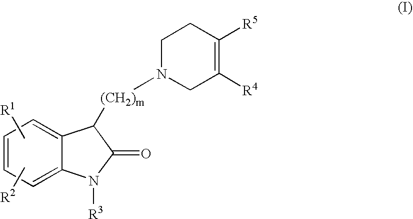

### TECHNICAL FIELD OF THE INVENTION

The invention relates to new 3-substituted indol-2-one derivatives, a process for the preparation thereof, pharmaceutical compositions containing said new indol-2-one derivatives and the use of said compounds for the treatment of diseases.

More particularly the present invention is concerned with new 3,3-disubstituted indol-2-one derivatives of the general Formula (I), 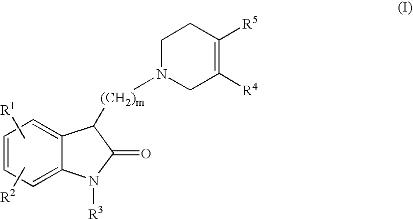
wherein
R¹ stands for hydrogen, halogen or alkyl having 1 to 7 carbon atom(s);
R² represents hydrogen or alkyl having 1 to 7 carbon atom(s);
R³ denotes hydrogen or alkyl having 1 to 7 carbon atom(s);
R⁴ represents hydrogen and R⁵ stands for a group of the general Formula (II), 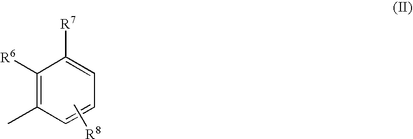
wherein R⁶, R⁷ and R⁸ each represents hydrogen, halogen, trifluoromethyl or straight or branched chain alkyl or alkoxy having 1 to 7 carbon atom(s), or R⁶ and R⁷ together form ethylene-dioxy, or
R⁴ and R⁵ form, together with the adjacent carbon atoms of the tetrahydropyridine ring, phenyl or a 5- or 6-membered heterocyclic ring containing a sulfur as heteroatom, which may optionally carry a halogen substituent;
m is 1, 2, 3 or 4;
and pharmaceutically acceptable acid addition salts thereof.

### TECHNICAL BACKGROUND OF THE INVENTION

U.S. Pat. No. 4,452,808 discloses 4-aminoalkyl-indol-2-one derivatives having a selective D₂ receptor activity. These compounds can be used for the treatment of hypertension. One of the compounds provided by this patent, namely 4-[2-(di-N-propylamino)ethyl]-2(3H)-indolone, is used for the treatment of Parkinson's disease.

European patent No. 281,309 provides indol-2-one derivatives carrying an arylpiperazinyl-alkyl substituent in position 5, which can be applied for the treatment of psychotic conditions. One of the compounds described in this patent, namely 5-[2-[4-(1,2-benzisothiazol-3-yl)-1-piperazinyl]-ethyl]-6-chloro-1,3-dihydro-2H-indol-2-one, exerts its activity by interaction with D₂, 5-HT_{1A} and 5-HT₂ receptors and is used in the clinical treatment as an antipsychotic agent.

European patent No. 376,607 discloses indol-2-one derivatives substituted in position 3 by an alkylpiperazinyl-aryl group, which exert their activity on 5-HT_{1A} receptors and are useful for the treatment of central nervous disorders.

In the international patent application WO 98/008816 indol-2-one derivatives containing a substituted alkylpiperazinyl, substituted alkyl-piperidinyl or alkyl-cyclohexyl group in position 3 are disclosed. These compounds exert anti-psychotic activity.

The acceleration of technical-social development in the XX. century constitutes a permanent compulsion of adaptation for humans, which, in adverse cases, my lead to the occurrence of adaptation disorders. Adaptation disorders constitute an important risk factor in the development of diseases of mental or psycho-somatic origin, such as anxiolytic syndrome, stress disorder, depression, schizophrenia, gastrointestinal diseases or cardiovascular diseases.

Beside the difficulties during adaptation to the environment another great problem of modern societies is the rapid ageing of population. Owing to the results of modern medical science life expectancy has increased, and the diseases occurring due to ageing or developing in the declining years, particularly the number of mental diseases has grown in leaps and bounds. The solution of the treatment of Alzheimer's disease, vascular dementias and senile dementia has become a social problem. For the treatment of the above clinical patterns most widespreadly pharmaceuticals exerting their activity on the benzodiazepine system (e.g. diazepam) or on central 5-HT_{1A} receptors (e.g. buspiron, ziprasidon) have been applied. In case of psychosomatic diseases anxiolytic therapy is often complemented by the administration of pharmaceuticals possessing antihypertensive (acting on the α₁ or α₂ receptor), or antiulcer (H₁-receptor antagonist) activity.

Anxiolytics of benzodiazepine type have several unpleasant side-effects. They cause decline of the power of concentration and memory and possess muscle relaxant effect. Said side-effects influence the quality of life of the patients in an adverse manner restricting the scope of application of such pharmaceuticals.

The pharmaceuticals acting on 5-HT_{1A} receptors that have been so far applied in the therapy are accompanied, however, by several drawbacks and undesired side-effects. It is a drawback that the anxiolytic effect can be achieved only after a treatment lasting for at least 10-14 days. Besides, after the initial administration an anxiogenic effect occurs. As to the side-effects, the occurrence of sleepiness, somnolence, vertigo, hallucination, headache, cognitive disorder or nausea has often been observed.

### SUMMARY OF THE INVENTION

The object of the present invention is to develop pharmaceutical ingredients which are devoid of the above-specified drawbacks and undesired side-effects characteristic of the active agents binding to 5-HT_{1A} receptors and which, at the same time, can be used for the treatment of disorders of the central nervous system.

The invention is based on the surprising recognition that the 3-alkyl substituted indol-2-one derivatives of the general Formula (I) considerably bind to 5-HT₇ receptors and inhibit serotonin uptake.

### DETAILED DESCRIPTION OF THE INVENTION

According to an aspect of the present invention there are provided novel 3-substituted indol-2-on derivatives of the general Formula (I), wherein

R¹ stands for hydrogen, halogen or alkyl having 1 to 7 carbon atom(s);

R² represents hydrogen or alkyl having 1 to 7 carbon atom(s);

R³ denotes hydrogen or alkyl having 1 to 7 carbon atom(s);

R⁴ represents hydrogen and R⁵ stands for a group of the general Formula (II), 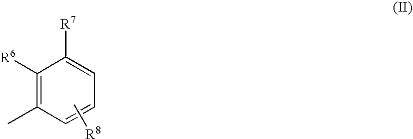
wherein R⁶, R⁷ and R⁸ each represents hydrogen, halogen, trifluoromethyl or straight or branched chain alkyl or alkoxy having 1 to 7 carbon atom(s), or R⁶ and R⁷ together form an ethylene-dioxy group, or
R⁴ and R⁵ form, together with the adjacent carbon atoms of the tetrahydropyridine ring, phenyl or a 5- or 6-membered heterocyclic ring containing a sulfur as heteroatom, which may optionally carry a halogen substituent;
m is 1, 2, 3 or 4;
and pharmaceutically acceptable acid addition salts thereof.

The term “alkyl” used throughout this specification is intended to mean straight or branched chain, saturated alkyl groups having 1 to 7, preferably 1 to 4 carbon atom(s), (e.g. methyl, ethyl, 1-propyl, 2-propyl, n-butyl, isobutyl or tert. butyl group etc.)

The term “halogen” encompasses all the four halogen atoms, such as fluorine, chlorine, iodine and bromine, and preferably stands for chlorine or bromine.

The term “leaving group” relates to an alkylsulfonyloxy or arylsulfonyloxy group, such as methylsulfonyloxy, or p-toluenesulfonyloxy group; or a halogen atom, preferably bromine or chlorine.

The term “pharmaceutically acceptable acid addition salts” relates to non-toxic salts of the compounds of the general Formula (I) formed with pharmaceutically acceptable organic or inorganic acids. Inorganic acids suitable for salt formation are e.g. hydrogen chloride, hydrogen bromide, phosphoric, sulfuric or nitric acid. As organic acids formic, acetic, propionic, maleic, fumaric, succinic, lactic, malic, tartaric, citric, ascorbic, malonic, oxalic, mandelic, glycolic, phtalic, benzenesulfonic, p-toluenesulfonic, naphthalic or methanesulfonic acids can be used. Furthermore, carbonates and hydro-carbonates are also considered as pharmaceutically acceptable salts.

According to a further aspect of the present invention there is provided a process for the preparation of the compounds of general Formula (I) and pharmaceutically acceptable acid addition salts thereof, which comprises
(a) reacting a compound of the general Formula (III), 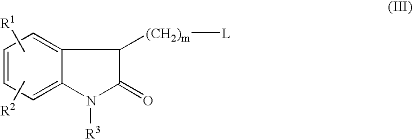
wherein L stands for hydroxy, R¹, R², R³ and m are as stated above, with an arylsulfonyl chloride or with a straight or branched chain C₁₋₇ alkylsulfonyl chloride in the presence of an organic base, and reacting the thus-obtained compound of the general Formula (III), wherein L represents aryl or alkylsulfonyloxy, with a pyridine derivative of the general Formula (IV, 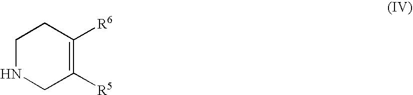
wherein R⁵ and R⁶ are as stated above, in the presence of an acid binding agent, or
   (b) reacting a compound of the general Formula (V), 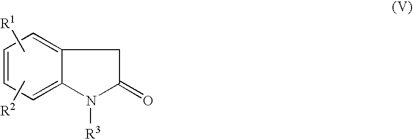
wherein R¹, R² and R³ are as stated above, with a compound of the general Formula (VII), 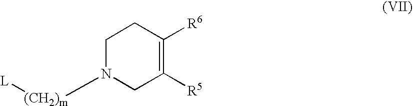
wherein R⁵, R⁶ and m are as stated above, in the presence of a strong base.

If desired, the compound of the general Formula (I), wherein R² stands for hydrogen obtained according to any of the above variants is halogenated or the free base is liberated from the salt thereof or converted into a pharmaceutically acceptable acid addition salt thereof.

The compounds of the general Formula (I), wherein R¹-R⁵ and m are as stated above, can be prepared by reacting a compound of the general Formula (III), wherein R¹-R³ and m are as stated above and L is a leaving group, with a compound of the general Formula (IV), wherein R⁴-R⁵ are as stated above, by methods known from the literature [Houben-Weyl: Methoden der organischen Chemie, Georg Thieme Verlag, Stuttgart, 1992, 4^{th} Edition, vol. E16d (ed.: D. Klamann); R. C. Larock: Comprehensive Organic Transformations, 2. ed., John Wiley & Sons, New York, 1999, 789; D. A. Walsh, Y-H, Chen, J. B. Green, J. C. Nolan, J. M. Yanni J. Med. Chem. 1990, 33, 1823-1827].

During the preparation of the compounds of the general Formula (III) the formation of the substituents can be carried out in optional succession according to methods known from the literature. It is expedient to prepare the compounds of the general Formula (III) by reacting a compound of the general Formula (V)—wherein L and n are as stated above and L′ is a leaving group or a group that can be converted into a leaving group—with a compound of the general Formula (VI),
L-(CH₂)ₘ-L′ (VI)
wherein R¹-R⁴ are as stated above, which has been prepared according to methods known from the literature [Houben-Weyl: Methoden der organischen Chemie, Georg Thieme Verlag, Stuttgart, 1977, 4^{th} Edition, vol. V/2b; A. R. Katritzky, Ch. W. Rees: Comprehensive Heterocyclic Chemistry, 1^{th} Edition, Pergamon, Oxford, 1984, vol. 4. (ed.: C. W. Bird, G. W. H. Cheeseman), 98-150 and 339-366; G. M. Karp Org. Prep. Proc. Int. 1993, 25, 481-513; B. Volk, T. Mezei, Gy. Simig Synthesis 2002, 595-597].

The compounds of the general Formula (I), wherein R¹-R⁵ and m are as stated above, can also be prepared by reacting a compound of the general Formula (V), wherein R¹-R³ are as stated above, with a compound of the general Formula (VII), wherein R⁴-R⁵ and m are as stated above and L is a leaving group, by methods known from the literature [R. J. Sundberg: The chemistry of indoles, Academic Press, New York, 1970, vol. VII.; A. R. Katritzky, Ch. W. Rees: Comprehensive Heterocyclic Chemistry, 1^{th} Edition, Pergamon, Oxford, 1984, vol. 4. (ed.: C. W. Bird, G. W. H. Cheeseman), 98-150 and 339-366; G. M. Karp Org. Prep. Proc. Int. 1993, 25, 481-513; A. S. Kende, J. C. Hodges Synth. Commun. 1982, 12, 1-10; W. W. Wilkerson, A. A. Kergaye, S. W. Tam J. Med. Chem. 1993, 36, 2899-2907].

The compounds of the general Formula (I), wherein R¹-R⁵ and n are as stated above, can also be prepared by carrying out the formation of the substituents R¹-R⁸ in different succession in the last reaction step. In this case a compound of the general Formula (I) is used as starting substance wherein all substituents are as stated above except the one to be formed, which can be any one selected from R¹, R², R³, R⁴, R⁵, R⁶, R⁷ and R⁸. The introduction and conversion of the substituents are carried out according to methods known from the literature [Houben-Weyl: Methoden der organischen Chemie, Georg Thieme Verlag, Stuttgart, 1977, 4^{th} Edition, IV/1a-d; vol. V/2b]. During the introduction of the substituents application or elimination of protecting groups may become necessary. Such methods are specified in T. W. Greene, Protective groups in organic synthesis, John Wiley & Sons, 1981.

The compounds of the general Formula (I), wherein R¹-R⁵ and n are as stated above, can also be prepared by carrying out the formation of the substituents R¹-R⁸ in different succession in the last reaction step. In this case as starting substance a compound of the general Formula (I) is applied wherein all substituents are as stated above except the one to be formed, which can be any one selected from R¹, R², R³, R⁴, R⁵, R⁶, R⁷ or R⁸. The introduction or conversion of the substituents can be carried out by methods analogous to those known from the literature [Houben-Weyl: Methoden der organischen Chemie, Georg Thieme Verlag, Stuttgart, 1977, 4^{th} Edition, IV/1a-d; vol. V/2b]. During the introduction of substituents the application and removal of protecting groups may be necessary. Such processes are specified in T. W. Greene, Protective groups in organic synthesis, John Wiley & Sons, 1981.

The compounds of the general Formulae (IV), (V), (VI) and (VII) are known from the literature or can be produced by analogous methods.

According to a further aspect of the present invention there are provided pharmaceutical compositions comprising as active ingredient a compound of the general Formula (I) or a pharmaceutically acceptable acid addition salt thereof in admixture with one or more conventional carrier(s) or auxiliary agent(s).

The pharmaceutical compositions according to the present invention contain generally 0.1-95% by weight, preferably 1-50% by weight, particularly 5-30% by weight of the active ingredient.

The pharmaceutical compositions of the present invention may be suitable for oral (e.g. powders, tablets, coated tablets, capsules, microcapsules, pills, solutions, suspensions or emulsions), parenteral (e.g. injection solutions for intravenous, intramuscular, subcutaneous or intraperitoneal use), rectal (e.g. suppositories) transdermal (e.g. plasters) or local (e.g. ointments or plasters) administration or for the application in form of implants. The solid, soft or liquid pharmaceutical compositions according to the invention may be produced by methods conventionally applied in the pharmaceutical industry. The solid pharmaceutical compositions for oral administration containing the compounds of the general Formula (I) or pharmaceutically acceptable acid addition salts thereof may comprise fillers or carriers (such as lactose, glucose, starch, potassium phosphate, micro-crystalline cellulose), binding agents (such as gelatine, sorbite, polyvinyl pyrrolidone), disintegrants (such as croscarmelose, Na-carboxy-methyl cellulose, crospovidone), tabletting auxiliary agents (such as magnesium stearate, talc, polyethylene glycol, silicic acid, silicon dioxide) and surface-active agents (e.g. sodium lauryl sulfate).

The liquid compositions suitable for oral administration can be solutions, suspensions or emulsions. Such compositions may contain suspending agents (e.g. gelatine, carboxymethyl cellulose), emulsifiers (e.g. sorbitane monooleate, solvents (e.g. water, oils, glycerol, propylene glycol, ethanol), buffering agents (e.g. acetate, phosphate, citrate buffers) or preservatives (e.g. methyl-4-hydroxybenzoate).

Liquid pharmaceutical compositions suitable for parenteral administration are generally sterile isotonic solutions optionally containing, in addition to the solvent, buffering agents or preservatives.

Soft pharmaceutical compositions containing as active ingredient a compound of the general Formula (I) or a pharmaceutically acceptable acid addition salt thereof, such as suppositories, contain the active ingredient evenly dispersed in the basic material of the suppository (e.g. in polyethylene glycol or cocoa butter).

The pharmaceutical compositions according to the present invention can be prepared by known methods of the pharmaceutical industry. The active ingredient is admixed with pharma-ceutically acceptable solid or liquid carriers and/or auxiliary agents and the mixture is brought to galenic form. The carriers and auxiliary agents together with the methods which can be used in the pharmaceutical industry are disclosed in the literature (Remington's Pharmaceutical Sciences, Edition 18, Mack Publishing Co., Easton, USA, 1990).

The pharmaceutical compositions according to the present invention contain generally a dosage unit. The daily dosage for human adults can be generally 0.1-1000 mg/kg body weight of a compound of the general Formula (I) or a pharmaceutically acceptable acid addition salts there-of. Said daily dose can be administered in one or more portion(s). The actual daily dose depends on several factors and is determined by the physician.

According to a further aspect of the present invention there is provided the use of the compounds of the general Formula (I) or pharmaceutically acceptable acid addition salts thereof for the treatment or prophylaxis of central nervous disorders, particularly depression, anxiety, compulsive disorder, panic disease, social phobia, schizophrenia, mood disorders, mania, mental decline, stroke, cell death in certain areas of the central nervous system, mental decline followed by cerebellar cell death, Alzheimer's disease, dementia, post-traumatic disease or stress disease.

The biological activity of the compounds according to the invention has been demonstrated by receptor binding experiments.

Human cloned receptors or frontal cortex preparations of male Wistar rats weighing 120-200 g were used for the experiments. The protein contents of membrane preparations were determined according to the method of Lowry (1951).

In the course of 5-HT₇ receptor binding studies the applied tissue was CHO cell culture, the ligand was ³H-LSD, and for the non-specific binding clozapine (25 μM) was used as ligand. In the serotonin uptake experiment cortex was used as tissue. As ligand tritiated serotonin, as non-specifically binding ligand fluoxetine (100 μM) was applied.

IC₅₀ is the concentration where the difference between whole binding and non-specific binding in the presence of 10 μM serotonin creatinine sulfate is 50%. The compounds with an IC₅₀ value smaller than 100 nmol were considered effective in this test. The results of the experiments are shown in Tables 2 and 3.

TABLE 2
Inhibition of 5-HT₇ receptor binding

| | IC₅₀ |
|---|---|
| No. of Example | nmole |
| 5 | <100 |
| 6 | <100 |
| 7 | <100 |
| 8 | <100 |
| 9 | <100 |
| 10 | <100 |
| 11 | <100 |
| 12 | <100 |
| 13 | <100 |
| 14 | <100 |

TABLE 3
Inhibition of 5-HT uptake

| | IC₅₀ |
|---|---|
| No. of Example | nmole |
| 13 | <100 |
| 14 | <100 |

From the results of the above experiments it can be established that the test compounds considerably bind to 5-HT₇ receptors and inhibit serotonin uptake.

On the basis of the above experiments the compounds according to the invention seem to be suitable for the treatment or prophylaxis of the diseases listed above. The combination of the 5-HT₇ receptorial and serotonin uptake inhibiting effects is particularly surprising and opens up new possibilities in the therapy. This double point of attack renders the compounds particularly suitable for the treatment of compulsive disorder, panic disease and social phobia, which disorders are basically treated by the application of serotonin uptake inhibitors.

Further details of the present invention are provided in the following examples without limiting the scope of protection to said examples.

### Preparation of Mesyl Esters (Process “A”)

The 3-(4-hydroxybutyl)-oxindoles are prepared according to a method known from the literature [B. Volk, T. Mezei, Gy. Simig Synthesis 2002, 595; B. Volk, Gy. Simig Eur. J. Org. Chem. 2003, 18, 3991-3996].

55 mmoles of 3-(4-hydroxybutyl)-oxindole are dissolved in 150 ml of THF, 15.2 ml (110 mmoles) of triethyl amine are added to it, and the solution is cooled in an acetone-dry ice bath to −78° C. While stirring at the same temperature 8.5 ml (110 mmoles) of mesyl chloride are dropped to it and the solution is allowed to warm to room temperature. It is stirred at room temperature for 1 hour, the triethyl amine hydrochloride is filtered off, the filtrate is evaporated, the residue is taken up in ethyl acetate and extracted several times with 10% by volume hydrogen chloride solution so that the pH of the aqueous phase is acidic. The organic phase is dried over sodium sulfate, evaporated, the residual oil is crystallized by trituration with diisopropyl ether, stirred in 100 ml of di-isopropyl ether, filtered, washed with hexane and dried. The product is purified by recrystal-lization from the solvent indicated after the melting point of the given substance.

### EXAMPLE 1

### 3-(4-Mesyloxybutyl)-1,3-dihydro-2H-indol-2-one

The title compound is prepared according to process A starting from 3-(4-hydroxybutyl)-1,3-dihydro-2H-indol-2-one.

M.p.: 84-85° C. (heptane-ethyl acetate).

IR (KBr): 3180, 1705 (C═O) cm⁻¹.

¹H-NMR (CDCl₃, TMS, 400 MHz): 9.33 (1H, s), 7.22 (1H, d, J=7.1 Hz), 7.21 (1H, t, J=7.0 Hz), 7.03 (1H, t, J=7.5 Hz), 6.93 (1H, d, J=7.6 Hz), 4.19 (2H, t, J=6.5 Hz), 3.49 (1H, t, J=6.0 Hz), 2.97 (3H, s), 2.05-1.98 (2H, m), 1.82-1.72 (2H, m) 1.58-1.40 (2H, m) ppm.

¹³C-NMR (CDCl₃, TMS, 101 MHz): 180.5, 141.6, 129.1, 127.9, 123.9, 122.3, 109.9, 69.5, 45.7, 37.2, 29.6, 28.9, 21.6 ppm.

### EXAMPLE 2

### 5-Fluoro-3-(4-mesyloxybutyl)-1,3-dihydro-2H-indol-2-one

The title compound is prepared according to process A starting from 5-fluoro-3-(4-hydroxybutyl)-1,3-dihydro-2H-indol-2-one.

M.p.: 106-108° C. (hexane-ethyl acetate).

IR (KBr): 3169, 1702 (C═O), 1356, 1175 (SO₂) cm⁻¹.

¹H-NMR (CDCl₃, TMS, 500 MHz): 1.43-1.55 (2H, m), 1.73-1.83 (2H, m), 1.97-2.05 (2H, m), 2.99 (3H, s), 3.50 (1H, t, J=5.9 Hz), 4.21 (2H, dq, J=1.4, 6.3 Hz), 6.86 (1H, dd, J=4.3, 8.4 Hz), 6.93 (1H, dt, J=2.3, 9.0 Hz), 6.97 (1H, dd, J=2.0, 7.3 Hz), 9.22 (1H, s) ppm.

¹³C-NMR (CDCl₃, TMS, 125.6 MHz): 180.2, 158.9 (d, J=240.6 Hz), 137.5 (d, J=1.7 Hz), 130.8 (d, J=8.5 Hz), 114.3 (d, J=27.5 Hz), 111.9 (d, J=24.8 Hz), 110.4 (d, J=8.1 Hz), 69.4, 46.2, 37.3, 29.5, 28.9, 21.5 ppm.

### EXAMPLE 3

### 6-Fluoro-3-(4-mesyloxybutyl)-1,3-dihydro-2H-indol-2-one

The title compound is prepared according to process A starting from 6-fluoro-3-(4-hydroxybutyl)-1,3-dihydro-2H-indol-2-one.

M.p.: 106-108° C. (hexane-ethyl acetate).

IR (KBr): 3161, 1705 (C═O), 1335, 1313, 1167 (SO₂) cm⁻¹.

¹H-NMR (CDCl₃, TMS, 500 MHz): 1.46-1.51 (2H, m), 1.78 (2H, q, J=6.7 Hz), 2.00 (2H, q, J=8.1 Hz), 2.99 (3H, s), 3.46 (1H, t, J=5.9 Hz), 4.21 (2H, dt, J=1.5, 6.5 Hz), 6.68 (1H, dd, J=2.3, 8.8 Hz), 6.72 (1H, dt, J=2.3, 8.9 Hz), 7.15 (1H, dd, J=5.4, 8.1 Hz), 9.15 (1H, br s) ppm.

¹³C-NMR (CDCl₃, TMS, 125.6 MHz): 21.6, 28.9, 29.7, 37.3, 45.3, 69.5, 98.6 (d, J=27.4 Hz), 108.7 (d, J=22.5 Hz), 124.5 (d, J=3.0 Hz), 124.9 (d, J=9.5 Hz), 142.8 (d, J=11.8 Hz), 162.6 (d, J=244.6 Hz), 180.7 ppm.

### EXAMPLE 4

### 5-Methyl-3-(4-mesyloxybutyl)-1,3-dihydro-2H-indol-2-one

The title compound is prepared according to process A starting from 3-(4-hydroxybutyl)-5-methyl-1,3-dihydro-2H-indol-2-one.

M.p.: 89-90° C. (hexane-ethyl acetate).

IR (KBr): 3175, 1710 (C═O), 1351, 1176 (SO₂) cm⁻¹

¹H-NMR (CDCl₃, TMS, 400 MHz): 9.13 (1H, s), 7.03 (1H, s), 7.01 (1H, dd, J=7.9, 0.8 Hz), 6.81 (1H, d, J=7.9 Hz), 4.20 (2H, t, J=6.5 Hz), 3.45 (1H, t, J=5.9 Hz), 2.98 (3H, s), 2.33 (3H, s), 1.99 (2H, q, J=7.4 Hz), 1.79-1.75 (2H, m), 1.51-1.42 (2H, m) ppm.

¹³C-NMR (CDCl₃, TMS, 101 MHz): 180.4, 139.1, 131.7, 129.2, 128.2, 124.7, 109.5, 69.6, 45.8, 37.2, 29.6, 28.9, 21.5, 21.0 ppm.

### Coupling Reaction of Mesyl Esters with Bases (Process “B”)

The melt of the secondary amine (12 mmoles) is warmed to 120° C. under slow stirring, and the mesyl compound (12 mmoles) and sodium carbonate (1.36 g; 12 mmoles) are added to it at the same temperature. The mixture is allowed to react for 1 hour, the melt is allowed to cool, ethyl acetate and water are added to it and the phases are separated. The organic phase is evaporated, the residual oil is subjected to chromatography on a short column using ethyl acetate as eluent. As main products the desired compounds are obtained.

Processing method 1: If the product purified by column chromatography gets crystalline upon rubbing with diethyl ether, it is filtered off and recrystallized from a mixture of hexane and ethyl acetate. The desired compounds are obtained in form of white crystals.

Processing method 2: If the basic product does not get crystalline upon the addition of diethyl ether, it is dissolved in 200 ml of ether, the slight amount of floating precipitate is filtered off and to the pure solution the calculated amount (1 molar equivalent) of hydrogen chloride dissolved in ether diluted with 50 ml of diethyl ether is dropped under vigorous stirring. The separated white salt is filtered off, washed with ether and hexane and dried in a vacuum pistol at room temperature for 3 hours.

Processing method 3: If the basic product does not get crystalline upon the addition of diethyl ether and does not provide a well-filterable salt with hydrogen chloride, it is dissolved in 100 ml of hot ethyl acetate, and a solution of 1 molar equivalent of oxalic acid dihydrate in 30 ml of hot ethyl acetate is dropped to it within 10 minutes, under stirring. The white oxalate salt gets separated upon cooling. It is filtered off at room temperature, washed with ethyl acetate and hexane and dried.

### EXAMPLE 5

### 3-{4-[4-(3-Trifluoromethyl-phenyl)-1,2,3,6-tetrahydropyridin-1-yl]-butyl}-1,3-dihydro-2H-indol-2-one monooxalate

The title compound is prepared according to process B by applying processing method 3 starting from 3-(4-mesyloxybutyl)-1,3-dihydro-2H-indol-2-one and 4-(3-trifluoromethylphenyl)-1,2, 3,6-tetrahydro-pyridine.

M.p.: 159-161° C.

IR (KBr): 3421, 1706 (C═O), 1332, 1169, 1125 cm⁻¹.

¹H-NMR (DMSO-d₆, TMS, 400 MHz): 1.40-1.20 (2H, m), 1.75-1.64 (2H, m), 1.96-1.78 (2H, m), 2.77 (2H, br s), 3.03 (2H, t, J=8.0 Hz), 3.31 (2H, t, J=5.3 Hz), 3.46 (1H, t, J=5.9 Hz), 3.78 (2H, br s), 6.33 (1H, s), 6.84 (1H, d, J=7.6 Hz), 6.95 (1H, dt, J=0.8, 7.6 Hz), 7.18 (1H, t, J=7.7 Hz), 7.27 (1H, d, J=7.3 Hz), 7.62 (1H, t, J=7.7 Hz), 7.68 (1H, d, J=7.7 Hz), 7.77 (1H, s), 7.80-7.76 (1H, m) 9.5 (2H, br s), 10.4 (1H, s) ppm.

¹³C-NMR (DMSO-d₆, TMS, 101 MHz): 22.8, 23.9, 24.0, 29.6, 45.1, 48.1, 49.9, 54.8, 109.4, 119.4, 121.4, 121.5 (q, J=3.8 Hz), 124.2, 124.4 (q, J=272.5 Hz), 124.5 (q, J=3.4 Hz), 127.8, 129.1, 129.6 (q, J=31.7 Hz), 129.7, 129.9, 133.1, 139.9, 142.9, 164.6, 179.0 ppm.

Elemental analysis for the Formula C₂₆H₂₇F₃N₂O₅ (504.51):

Calculated: C, 61.90; H, 5.39; N, 5.55%.

Found: C, 61.50; H, 5.40; N, 5.52%.

### EXAMPLE 6

### 3-[4-(6,7-Dihydro-4H-thieno[3,2-c]pyridin-5-yl)-butyl]-1,3-dihydro-2H-indol-2-one monooxalate

The title compound is prepared according to process B by applying processing method 3 starting from 3-(4-mesyloxybutyl)-1,3-dihydro-2H-indol-2-one and 6,7-dihydro-4H-thieno[3,2-c]pyridine.

M.p.: 168-170° C.

IR (KBr): 1712 (C═O) cm⁻¹.

¹H-NMR (DMSO-d₆, TMS, 400 MHz): 1.25 (2H, br s), 2.0-1.6 (4H, br s), 3.06 (4H, br s), 3.39 (2H, br s)m 3.45 (1H, br s), 4.18 (2H, br s), 6.0-5.0 (2H, br s), 6.83 (1H, d, J=7.5 Hz), 6.88 (1H, d, J=4.7 Hz), 6.95 (1H, t, J=7.2 Hz), 7.17 (1H, t, J=7.3 Hz), 7.26 (1H, d, J=6.5 Hz), 7.44 (1H, d, J=4.8 Hz) ppm.

¹³C-NMR (DMSO-d₆, TMS, 101 MHz): 178.9, 164.0, 142.9, 131.7, 129.7, 129.7, 127.8, 125.4, 125.1, 124.2, 121.4, 109.4, 55.0, 50.6, 49.4, 45.1, 29.6, 24.0, 22.7, 22.2 ppm.

Elemental analysis for the Formula C₂₁H₂₄N₂O₅S (416.50):

Calculated: C, 60.56; H, 5.81; N, 6.73; S, 7.70%.

Found: C, 59.93; H, 5.86; N, 6.67; S, 7.58%.

### EXAMPLE 7

### 3-[4-(6,7-Dihydro-4H-thieno[3,2-c]pyridin-5-yl)-butyl]-5-fluoro-1,3-dihydro-2H-indol-2-one monohydrochloride

The title compound is prepared according to process B by applying processing method 2 starting from 5-fluoro-3-(4-mesyloxybutyl)-1,3-dihydro-2H-indol-2-one and 6,7-dihydro-4H-thieno[3,2-c]pyridine.

M.p.: 192-194° C.

IR (KBr): 3428, 1706 (C═O), 1187 cm⁻¹.

¹H-NMR (DMSO-d₆, TMS, 400 MHz): 1.34-1.24 (2H, m), 1.86-1.77 (4H, m), 3.07-3.19 (4H, br s), 3.27-3.39 (1H, br s), 3.51 (1H, t, J=5.6 Hz), 3.64 (1H, br s), 4.13 (1H, br s), 4.37 (1H, br s), 6.82 (1H, dd, J=4.5, 8.4 Hz), 6.89 (1H, d, J=5.1 Hz), 7.00 (1H, dt, J=2.4, 8.9 Hz), 7.20 (1H, dd, J=1.8, 8.3 Hz), 7.46 (1H, d, J=5.1 Hz) ppm.

¹³C-NMR (DMSO-d₆, TMS, 101 MHz): 21.8, 22.5, 23.5, 29.3, 45.6, 49.1, 50.1, 54.7, 109.9 (d, J=8.0 Hz), 112.1 (d, J=24.4 Hz), 113.0 (d, J=23.3 Hz), 125.3, 125.3, 128.4, 131.5 (d, J=10.7 Hz), 131.6, 139.2 (d, J=1.5 Hz), 158.1 (d, J=236.1 Hz), 178.7 ppm.

Elemental analysis for the Formula C₁₉H₂₂ClFN₂OS (380.92):

Calculated: C, 59.91; H, 5.82; Cl, 9.31; N, 7.35; S, 8.42%.

Found: C, 60.04; H, 5.81; Cl, 8.88; N, 7.25; S, 8.38%.

### EXAMPLE 8

### 3-[4-(2-Chloro-6,7-dihydro-4H-thieno[3,2-c]-pyridin-5-yl)-butyl]-1,3-dihydro-2H-indol-2-one monohydrochloride

The title compound is prepared according to process B by applying processing method 2 starting from 3-(4-mesyloxybutyl)-1,3-dihydro-2H-indol-2-one and 2-chloro-6,7-dihydro-4H-thieno-[3,2-c]pyridine.

M.p.: 103-106° C.

IR (KBr): 3421, 3168, 2565, 1707 (C═O), 754 cm⁻¹.

¹H-NMR (CDCl₃, TMS, 400 MHz): 1.40 (2H, m), 1.99 (4H, m), 3.49-2.90 (6H, m), 3.64 (1H, br s), 3.85 (1H, m), 4.43, 4.47 (′H, br s), 6.63 (1H, s), 6.92 (1H, d, J=7.7 Hz), 7.02 (1H, dt, J=1.0, 7.6 Hz), 7.18 (1H, d, J=7.1 Hz), 7.20 (1H, tt, J=1.0, 7.2 Hz), 8.56-8.60 (1H, br s), 12.8 (1H, br s) ppm.

¹³C-NMR (CDCl₃, TMS, 101 MHz): 179.7, 141.9, 130.3, 129.9, 128.8, 128.0, 125.8, 123.9, 123.7, 122.2, 110.1, 54.7, 49.8, 49.1, 45.4, 29.3, 23.8, 22.7, 21.2 ppm.

Elemental analysis for the Formula C₁₉H₂₂Cl₂N₂OS (397.37):

Calculated: C, 57.43; H, 5.58; Cl, 17.84; N, 7.05; S, 8.07%.

Found: C, 56.26; H, 5.67; Cl, 17.22; N, 6.58; S, 7.57%.

### EXAMPLE 9

### 3-[4-(6,7-Dihydro-4H-thieno[3,2-c]pyridin-5-yl)-butyl]-6-fluoro-1,3-dihydro-2H-indol-2-one mono-hydrochloride

The title compound is prepared according to process B by applying processing method 2 starting from 6-fluoro-3-(4-mesyloxybutyl)-1,3-dihydro-2H-indol-2-one and 6,7-dihydro-4H-thieno[3,2-c]pyridine.

M.p.: 194-197° C.

IR (KBr): 3160, 2566, 1710 (C═O) cm⁻¹.

¹H-NMR (DMSO-d₆, TMS, 400 MHz): 1.36-1.23 (2H, m), 1.95-1.78 (4H, m), 3.36-3.10 (4H, m), 3.39 (2H, br s), 3.46 (1H, t, J=5.9 Hz), 4.15 (1H, br s), 4.36 (1H, br s), 6.67 (1H, dd, J=2.4, 9.2 Hz), 6.75 (1H, dt, J=2.4, 9.1 Hz), 6.90 (1H, d, J=5.1 Hz), 7.29 (1H, dd, J=5.8, 8.0 Hz), 7.46 (1H, d, J=5.2 Hz), 10.6 (1H, s), 11.2 (1H, br s) ppm.

¹³C-NMR (DMSO-d₆, TMS, 101 MHz): 21.8, 22.6, 23.5, 29.6, 44.6, 49.1, 50.1, 54.7, 97.6 (d, J=27.1 Hz), 107.3 (d, J=22.1 Hz), 125.2, 125.3, 125.4, 125.5, 128.4, 131.6, 144.5 (d, J=12.2 Hz), 162.1 (d, J=240.7 Hz), 179.2 ppm.

Elemental analysis for the Formula C₁₉H₂₂ClFN₂OS (380.92):

Calculated: C, 59.91; H, 5.82; Cl, 9.31; N, 7.35; S, 8.42%.

Found: C, 59.67; H, 5.80; Cl, 9.03; N, 7.06; S, 8.18%.

### EXAMPLE 10

### 3-[4-(2-Chloro-6,7-dihydro-4H-thieno[3,2-c]-pyridin-5-yl)-butyl]-6-fluoro-1,3-dihydro-2H-indol-2-one monohydrochloride

The title compound is prepared according to process B by applying processing method 2 starting from 6-fluoro-3-(4-mesyloxybutyl)-1,3-dihydro-2H-indol-2-one and 2-chloro-6,7-dihydro-4H-thieno[3,2-c]pyridine.

M.p.: 214-216° C.

IR (KBr): 3413, 2560, 1710 (C═O) cm⁻¹.

¹H-NMR (DMSO-d₆, TMS, 400 MHz): 1.29 (2H, br s), 1.93-1.76 (4H, m), 3.35-2.98 (5H, m), 3.45 (1H, t, J=5.8 Hz), 3.68-3.63 (1H, m), 4.07-4.03 (1H, m), 4.34-4.28 (1H, m), 6.65 (1H, dd, J=2.4, 9.3 Hz), 6.75 (1H, dt, J=2.4, 9.1 Hz), 7.28 (1H, dd, J=5.9, 8.0 Hz), 10.6 (1H, s), 11.2 (1H, br s) ppm.

¹³C-NMR (DMSO-d₆, TMS, 101 MHz): 21.7, 22.5, 23.4, 29.5, 44.5, 48.7, 49.4, 54.6, 97.6 (d, J=27.1 Hz), 107.4 (d, J=22.1 Hz), 125.0, 125.4, 125.4 (d, J=8.4 Hz), 127.3, 128.1, 131.1, 144.5 (d, J=12.6 Hz), 162.1 (d, J=241.1 Hz), 179.2 ppm.

Elemental analysis for the Formula C₁₉H₂₁Cl₂FN₂OS (415.36):

Calculated: C, 54.94; H, 5.10; Cl, 17.07; N, 6.74; S, 7.72%.

Found: C, 53.76; H, 5.19; Cl, 16.50; N, 6.56; S, 7.76%

### EXAMPLE 11

### 3-[4-(2-Chloro-6,7-dihydro-4H-thieno[3,2-c]-pyridin-5-yl)-butyl]-5-fluoro-1,3-dihydro-2H-indol-2-one monohydrochloride

The title compound is prepared according to process B by applying processing method 2 starting from 5-fluoro-3-(4-mesyloxybutyl)-1,3-dihydro-2H-indol-2-one and 2-chloro-6,7-dihydro-4H-thieno[3,2-c]pyridine.

M.p.: 161-163° C.

IR (KBr): 3198, 2561, 1706 (C═O) cm⁻¹.

¹H-NMR (DMSO-d₆, TMS, 400 MHz): 1.40-1.20 (2H, m), 1.92-1.77 (4H, m), 3.01 (2H, m), 3.13 (2H, m), 3.30 (1H, m), 3.50 (1H, t, J=5.7 Hz), 3.65 (1H, m), 4.06 (1H, d, J=10.8 Hz), 4.33 (1H, d, 15.3 Hz), 6.82 (1H, dd, J=4.5, 8.4 Hz), 6.95 (1H, s), 7.00 (1H, dt, J=2.7, 9.1 Hz), 7.20 (1H, dd, J=1.8, 8.3 Hz) ppm.

¹³C-NMR (DMSO-d₆, TMS, 101 MHz): 21.7, 22.5, 23.4, 29.3, 45.6, 48.7, 49.4, 54.6, 110.0 (d, J=8.0 Hz), 112.1 (d, J=24.4 Hz), 114.0 (d, J=22.9 Hz), 125.0, 127.3, 128.1, 131.1, 131.5, 139.2, 158.1 (d, J=235.8 Hz), 178.8 ppm.

Elemental analysis for the Formula C₁₉H₂₁Cl₂FN₂OS (415.36):

Calculated: C, 54.94; H, 5.10; Cl, 17.07; N, 6.74; S, 7.72%.

Found: C, 54.64; H, 4.93; Cl, 16.42; N, 6.52; S, 7.52%.

### EXAMPLE 12

### 6-Fluoro-3-{4-[4-(3-trifluoromethyl-phenyl)-1,2,3,6-tetrahydropyridin-1-yl]-butyl}-1,3-dihydro-2H-indol-2-one monohydrochloride

The title compound is prepared according to process B by applying processing method 2 starting from 6-fluoro-3-(4-mesyloxybutyl)-1,3-dihydro-2H-indol-2-one and 4-(3-trifluoromethyl-phenyl)-1,2,3,6-tetrahydro-pyridine.

M.p.: 203-205° C.

IR (KBr): 3122, 2576, 1714 (C═O), 1336, 1136, 1120 cm⁻¹.

¹H-NMR (DMSO-d₆, TMS, 400 MHz): 1.35-1.29 (2H, m), 1.96-1.79 (4H, m), 2.84 (2H, br s), 3.11 (2H, t, J=7.8 Hz), 3.22 (2H, br s), 3.46 (1H, t, J=5.7 hz), 3.92-3.46 (3H, br s), 6.34 (1H, s), 6.68 (1H, dd, J=2.4, 9.3 Hz), 6.76 (1H, dt, J=2.4, 9.1 Hz), 7.29 (1H, dd, J=6.0, 7.4 Hz), 7.63 (1H, t, J=7.7 Hz), 7.77 (1H, s), 7.80 (1H, d, J=7.6 Hz), 10.6 (1H, br s), 11.1 (1H, br s) ppm.

¹³C-NMR (DMSO-d₆, TMS, 101 MHz): 22.6, 23.4, 23.6, 29.6, 44.6, 47.9, 49.4, 54.6, 97.6 (d, J=27.1 Hz), 107.4 (d, J=22.1 hz), 118.7, 121.5 (q, J=3.8 Hz), 124.4 (q, J=272.4 Hz), 124.6, 125.4, 125.5, 129.1, 129.6 (q, J=31.3 Hz), 129.9, 133.1, 139.6, 144.5 (d, J=1.2 Hz), 162.1 (d, J=240.7 Hz), 179.3 ppm.

Elemental analysis for the Formula C₂₄H₂₅ClF₄N₂O (468.93):

Calculated: C, 61.47; H, 5.37; Cl, 7.56; N, 5.97%.

Found: C, 60.89; H, 5.33; Cl, 7.46; N, 5.85%.

### EXAMPLE 13

### 3-{4-[4-(4-Chlorophenyl)-1,2,3,6-tetrahydro-pyridin-1-yl]-butyl}-1,3-dihydro-2H-indol-2-one

The title compound is prepared according to process B by applying processing method 1 starting from 3-(4-mesyloxybutyl)-1,3-dihydro-2H-indol-2-one and 4-(4-chlorophenyl)-1,2,3,6-tetra-hydropyridine.

M.p.: 122-124° C. (hexane-ethyl acetate).

IR (KBr): 3193, 1704 (C═O) cm⁻¹.

¹H-NMR (CDCl₃, TMS, 400 MHz): 1.46-1.38 (2H, m), 1.64-1.58 (2H, m), 2.04-1.95 (2H, m), 2.49 (2H, t, J=7.8 Hz), 2.54 (2H, br s), 2.73 (2H, t, J=5.6 Hz), 3.17 (2H, br s), 3.46 (1H, t, J=5.9 Hz), 6.01 (1H, t, J=1.7 Hz), 7.01 (1H, dt, J=0.9, 7.5 Hz), 7.18 (1H, t, J=7.7 Hz), 7.21 (1H, d, J=7.2 Hz), 7.29-7.23 (4H, m), 9.33 (1H, s) ppm.

¹³C-NMR (CDCl₃, TMS, 101 MHz): 23.7, 26.7, 27.5, 30.3, 45.9, 49.9, 52.7, 57.7, 109.8, 121.6, 122.1, 124.0, 126.1, 127.8, 128.3, 129.6, 132.7, 134.0, 138.9, 141.8, 180.6 ppm.

Elemental analysis for the Formula C₂₃H₂₅ClN₂O (380.92):

Calculated: C, 72.52; H, 6.62; Cl, 9.31; N, 7.35%.

Found: C, 72.08; H, 6.63; Cl, 9.07; N, 7.23%.

### EXAMPLE 14

### 5-Fluoro-3-{4-[4-(3-trifluoromethyl-phenyl)-1,2,3,6-tetrahydropyridin-1-yl]-butyl}-1,3-dihydro-2H-indol-2-one monohydrochloride

The title compound is prepared according to process B by applying processing method 2 starting from 5-fluoro-3-(4-mesyloxybutyl)-1,3-dihydro-2H-indol-2-one and 4-(3-trifluoromethyl-phenyl)-1,2,3,6-tetrahydro-pyridine.

M.p.: 201-204° C.

IR (KBr): 3243, 1706 (C═O), 1331, 1162, 1113 cm⁻¹.

¹H-NMR (DMSO-d₆, TMS, 400 MHz): 1.31-1.17 (2H, m), 2.00-1.78 (4H, m), 2.90-2.76 (2H, m), 3.12 (2H, br s), 3.21-3.18 (1H, m), 3.51 (1H, t, J=5.6 Hz), 3.99-3.58 (3H, m), 6.34 (1H, s), 6.83 (1H, dd, J=4.6, 8.5 Hz), 7.01 (1H, dt, J=2.5, 9.1 Hz), 7.21 (1H, d, J=6.8 Hz), 7.63 (1H, t, J=7.6 Hz), 7.69 (1H, d, J=7.6 Hz), 7.78 (1H, s), 7.80 (1H, d, J=7.6 Hz), 10.46 (1H, s), 11.0 (1H, br s) ppm.

¹³C-NMR (DMSO-d₆, TMS, 101 MHz): 22.5, 23.4, 23.5, 45.6, 47.9, 49.4, 54.5, 109.9 (d, J=8.4 Hz), 112.1 (d, J=24.8 Hz), 113.9 (d, J=23.3 Hz), 118.6, 121.5 (q, J=3.8 Hz), 124.3 (q, J=272.4 Hz), 124.6, 129.1, 129.6 (q, J=31.7 Hz), 129.9, 131.5 (d, J=8.4 Hz), 133.1, 139.1, 139.6, 158.1 (d, J=235.8 Hz), 178.7 ppm.

Elemental analysis for the Formula C₂₄H₂₅ClF₄N₂O (468.93):

Calculated: C, 61.47; H, 5.37; Cl, 7.56; N, 5.97%.

Found: C, 60.91; H, 5.38; Cl, 7.48; N, 5.93%.

### EXAMPLE 15

### 3-[4-(3,4-Dihydro-1H-isoquinolin-2-yl)-butyl]-1,3-dihydro-2H-indol-2-one monohydrochloride

The title compound is prepared according to process B by applying processing method 2 starting from 3-(4-mesyloxybutyl)-1,3-dihydro-2H-indol-2-one and 3,4-dihydro-1H-isoquinoline.

M.p.: 98-100° C.

IR (KBr): 3421, 2571, 1709 (C═O) cm⁻¹.

¹H-NMR (DMSO-d₆, TMS, 400 MHz): 1.40-1.27 (2H, m), 1.99-1.78 (4H, m), 3.1 (4H, t, J=8.0 Hz), 3.5-2.8 (2H, m), 3.47 (1H, t, J=5.9 Hz), 4.30 (2H, br s), 6.85 (1H, d, J=7.7 Hz), 6.96 (1H, t, J=7.3 Hz), 7.29-7.15 (6H, m), 10.4 (1H, s), 11.2 (1H, br s) ppm.

¹³C-NMR (DMSO-d₆, TMS, 101 MHz): 22.5, 23.2, 24.8, 29.4, 44.8, 48.9, 51.4, 54.8, 109.2, 121.2, 124.0, 126.5, 127.5, 127.6, 128.5, 128.6, 129.5, 131.5, 142.8, 178.7 ppm.

Elemental analysis for the Formula C₂₁H₂₅ClN₂O (356.90):

Calculated: C, 70.67; H, 7.06; Cl, 9.93; N, 7.85%.

Found: C, 68.92; H, 7.16; Cl, 9.63; N, 7.68%.

### EXAMPLE 16

### 3-[4-(2-Chloro-6,7-dihydro-4H-thieno[3,2-c]-pyridin-5-yl)-butyl]-5-methyl-1,3-dihydro-2H-indol-2-one monohydrochloride

The title compound is prepared according to process B by applying processing method 2 starting from 3-(4-chlorobutyl)-3-ethyl-5-methyl-1,3-dihydro-2H-indol-2-one and 2-chloro-6,7-dihydro-4H-thieno[3,2-c]pyridine.

M.p.: 109-114° C.

IR (KBr): 3185, 2566, 1705 (C═O) cm⁻¹.

¹H-NMR (DMSO-d₆, TMS, 400 MHz): 1.31-1.23 (2H, m), 1.92-1.76 (4H, m), 2.26 (3H, s), 3.00 (1H, d, J=16.9 Hz), 3.14 (3H, m), 3.38-3.27 (1H, m), 3.67-3.64 (1H, m), 4.05 (1H, dd, J=6.9, 14.6 Hz), 4.32 (1H, d, J=15.2 Hz), 6.72 (1H, d, J=7.8 Hz), 6.94 (1H, s), 6.97 (1H, dq, J=0.8, 7.8 Hz), 7.09 (1H, s), 10.31 (1H, s), 11.3 (1H, br s) ppm.

¹³C-NMR (DMSO-d₆, TMS, 101 MHz): 20.9, 21.7, 22.7, 23.5, 29.6, 45.1, 48.7, 49.4, 54.6, 109.1, 124.8, 125.0, 127.3, 128.0, 128.1, 129.7, 130.2, 131.1, 140.5, 178.8 ppm.

Elemental analysis for the Formula C₂₀H₂₄Cl₂N₂OS (411.40):

Calculated: C, 58.39; H, 5.88; Cl, 17.24; N, 6.81; S, 7.79%.

Found: C, 56.54; H, 6.11; Cl, 15.64; N, 6.43; S, 7.20%.

### EXAMPLE 17

### 6-Fluoro-3-{4-[4-(4-fluorophenyl)-3,6-dihydro-2H-pyridin-1-yl]-butyl}-1,3-dihydro-2H-indol-2-one monohydrochloride

The title compound is prepared according to process B by applying processing method 2 starting from 6-fluoro-3-(4-mesyloxybutyl)-1,3-dihydro-2H-indol-2-one and 4-(4-fluorophenyl)-1,2,3,6-tetrahydropyridine.

M.p.: 176-178° C.

IR (KBr): 3123, 2573, 1717 (C═O) cm⁻¹.

¹H-NMR (CDCl₃, TMS, 400 MHz): 1.39-1.25 (2H, m), 2.05-1.90 (4H, m), 4.2-2.5 (8H, m), 3.38 (1H, t, J=5.4 Hz), 5.93 (1H, s), 6.67 (1H, dt, J=2.3, 8.9 Hz), 6.73 (1H, dd, J=2.2, 8.8 Hz), 7.02 (2H, t, J=8.6 Hz), 7.09 (1H, dd, J=5.3, 8.1 Hz), 7.33 (H, dd, J=5.3, 8.9 Hz), 9.32 (1H, br s) ppm.

¹³C-NMR (CDCl₃, TMS, 101 MHz): 22.7, 23.8, 23.9, 29.4, 44.9, 48.5, 49.8, 55.1, 98.7 (d, J=27.5 Hz), 108.5 (d, J=22.5 Hz), 114.4, 115.5 (d, J=21.8 Hz), 124.2 (d, J=3.1 Hz), 124.8 (d, J=9.9 Hz), 126.8 (d, J=8.0 Hz), 134.3 (d, J=3.1 Hz), 135.0, 143.2 (d, J=12.2 Hz), 162.7 (d, J=244.1 Hz), 162.7 (d, J=248.7 Hz), 179.8 ppm.

Elemental analysis for the Formula C₂₃H₂₅ClF₂N₂O (418.92):

Calculated: C, 65.95; H, 6.02; Cl, 8.46; N, 6.69%

Found: C, 65.42; H, 6.15; Cl, 8.60; N, 6.72%.

### EXAMPLE 18

### 3-[4-(4-phenyl-3,6-dihydro-2H-pyridine-1-yl)-butyl]-1,3-dihydro-2H-indol-2-one

The title compound is prepared according to process B using processing method 1 starting from 3-(4-mesyloxy-butyl)-1,3-dihydro-2H-indol-2-one and 4-phenyl-1,2,3,6-tetrahydro-pyridine.

Melting point, 121-126° C.

IR (KBr): 3191, 1704 (C═O) cm⁻¹.

¹H-NMR (DMSO-d₆, TMS, 500 MHz): 1.34-1.22 (2H, m), 1.49-1.42 (2H, m), 1.85-1.77 (1H, m), 1.94-1.87 (1H, m), 2.32 (2H, t, J=7.3 Hz), 2.42 (2H, s), 2.55 (2H, t, J=5.6 Hz), 3.00 (2H, d, J=2.4 Hz), 3.43 (1H, t, J=5.6 Hz), 6.11 (1H, s), 6.82 (1H, d, J=7.4 Hz), 6.94 (1H, t, J=7.3 Hz), 7.16 (1H, t, J=7.5 Hz), 7.25-7.21 (2H, m), 7.32 (2H, t, J=7.8 Hz), 7.41 (2H, d, J=7.3 Hz), 10.35 (1H, s) ppm.

¹³C-NMR (DMSO-d₆, TMS, 125.6 MHz): 23.4, 26.7, 27.6, 29.9, 45.3, 50.1, 52.9, 57.6, 109.3, 121.4, 122.2, 124.1, 124.6, 127.1, 127.7, 128.5, 129.9, 134.1, 140.3, 142.9, 179.1 ppm.

Elemental analysis for the Formula C₂₃H₂₆N₂O (346.48)

Calculated: C, 79.73; H, 7.56; N, 8.09%.

Measured: C, 78.64; H, 7.43; N, 8.07%.

### EXAMPLE 19

### 3-{4-[4-(3-chlorophenyl)-3,6-dihydro-2H-pyridine-1-yl]-butyl}-1,3-dihydro-2H-indol-2-one monohydrochloride

The title compound is prepared according to process B using processing method 2 starting from 3-(4-mesyloxy-butyl)-1,3-dihydro-2H-indol-2-one and 4-(3-chlorophenyl)-1,2,3,6-tetrahydro-pyridine.

Melting point, 92-95° C.

IR (KBr): kb. 3150, 2574, 1708 (C═O), 1100 cm⁻¹.

¹H-NMR (DMSO-d₆, TMS, 500 MHz): 1.34-1.26 (2H, m), 1.74 (2H, sz), 1.93-1.80 (2H, m), 2.75 (2H, sz), 3.06 (2H, sz), 3.40-3.10 (2H, sz), 3.46 (1H, t, J=6.0 Hz), 3.7 (2H, sz), 6.27 (1H, s), 6.83 (1H, d, J=7.7 Hz), 6.96 (1H, dt, J=1.0, 7.6 Hz), 7.18 (1H, tt, J=0.9, 7.6 Hz), 7.27 (1H, d, J=7.3 Hz), 7.38 (1H, td, J=1.7, 7.7 Hz), 7.41 (1H, t, J=7.6 Hz), 7.45 (1H, td, J=1.6, 7.5 Hz), 7.53 (1H, t, J=1.6 Hz), 10.40 (1H, s), 10.6 (1H, sz) ppm.

¹³C-NMR (DMSO-d₆, TMS, 125.6 MHz): 22.7, 23.6, 23.8, 29.6, 45.1, 48.0, 49.6, 54.8, 109.4, 121.4, 123.7, 124.2, 124.9, 127.8, 127.8, 129.7, 130.5, 133.1, 133.6, 140.8, 143.0, 178.9 ppm.

Elemental analysis for the Formula C₂₃H₂₆Cl₂N₂O (417.38)

Calculated: C, 66.19; H, 6.28; Cl, 16.99; N, 6.71%.

Measured: C, 64.97; H, 6.58; Cl, 16.27; N, 6.51%.

### EXAMPLE 20

### 3-{4-[4-(3-chlorophenyl)-3,6-dihydro-2H-pyridine-1-yl]-butyl}-6-fluoro-1,3-dihydro-2H-indol-2-one monohydrochloride

The title compound is prepared according to process B using processing method 2 starting from 6-fluoro-3-(4-mesyloxybutyl)-1, 3-dihydro-2H-indol-2-one and 4-(3-chlorophenyl)-1,2, 3,6-tetrahydro-pyridine.

Melting point, 147-149° C.

IR (KBr): 3144, 2576, 1716 (C═O) cm⁻¹.

¹H-NMR (DMSO-d₆, TMS, 500 MHz): 1.34-1.25 (2H, m), 1.95-1.78 (4H, m), 3.93-2.74 (9H, m), 6.27 (1H, s), 6.78-6.27 (2H, m), 7.54-7.28 (5H, m), 10.63 (1H, s), 11.07 (1H, sz) ppm.

¹³C-NMR (DMSO-d₆, TMS, 125.6 MHz): 22.6, 23.4, 23.6, 29.6, 44.6, 47.9, 49.4, 54.6, 97.6 (d, J=26.4 Hz), 107.4 (d, J=22.5 Hz), 118.1, 123.7, 124.9, 125.5, 127.9, 130.6, 133.1, 133.7, 140.7, 144.5 (d, J=12.2 Hz), 162.1 (d, J=241.2 Hz), 179.3 ppm.

Elemental analysis for the Formula C₂₃H₂₅Cl₂FN₂O (435.37)

Calculated: C, 63.45; H, 5.79; Cl, 16.29; N, 6.43%.

Measured: C, 61.93; H, 5.98; Cl, 16.24; N, 5.98%.

### EXAMPLE 21

### 3-{4-[4-(3-chlorophenyl)-3,6-dihydro-2H-pyridine-1-yl]-butyl}-5-fluoro-1,3-dihydro-2H-indol-2-one monohydrochloride

The title compound is prepared according to process B by processing method 2 using 5-fluoro-3-(4-mesyloxy-butyl)-1,3-dihydro-2H-indol-2-one and 4-(3-chlorophenyl)-1,2,3,6-tetrahydro-pyridine as starting compounds.

Melting point, 96-101° C.

IR (KBr): 3391, 2580, 1705 (C═O) cm¹.

¹H-NMR (DMSO-d₆, TMS, 500 MHz): 1.33-1.28 (2H, m), 1.95-1.76 (4H, m), 2.74 (1H, m), 2.86 (1H, m), 3.18-3.09 (3H, m), 3.51 (1H, t, J=5.8 Hz), 3.57 (1H, m), 3.73 (1H, m), 3.94 (1H, m), 6.27 (1H, s), 6.83 (1H, m), 7.01 (1H, m), 7.22 (1H, m), 7.47-7.37 (3H, m), 7.53 (1H, s), 10.5 (1H, s), 11.0 (1H, sz) ppm.

Elemental analysis for the Formula C₂₃H₂₅Cl₂FN₂O (435.37)

Calculated: C, 63.45; H, 5.79; Cl, 16.29; N, 6.43%.

Measured: C, 63.25; H, 5.70; Cl, 15.85; N, 6.51%.

1. 3-Alkyl indol-2-one derivatives of the general Formula (I), 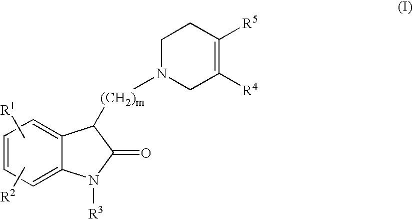
wherein
R¹ stands for hydrogen, halogen or alkyl having 1 to 7 carbon atom(s);
R² represents hydrogen or alkyl having 1 to 7 carbon atom(s);
R³ denotes hydrogen or alkyl having 1 to 7 carbon atom(s);
R⁴ represents hydrogen and R⁵ stands for a group of the general Formula (II), 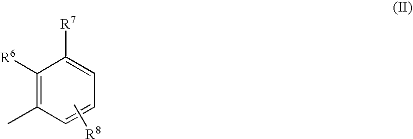
wherein R⁶, R⁷ and R⁸ each represents hydrogen, halogen, trifluoromethyl or straight or branched chain alkyl or alkoxy having 1 to 7 carbon atom(s), or R⁶ and R⁷ together form ethylenedioxy, or R⁶ and R⁷ together form ethylenedioxy; or R⁴ and R⁵ form, together with the adjacent carbon atoms of the tetrahydropyridine ring, phenyl or a 5- or 6-membered heterocyclic ring containing a sulfur as heteroatom, which may optionally carry a halogen substituent;
m is 1, 2, 3 or 4;
and pharmaceutically acceptable acid addition salts thereof.

2. 3-Alkyl indol-2-one derivatives of the general Formula (I),
wherein
R¹ represents hydrogen, halogen or alkyl having 1 to 7 carbon atom(s);
R² denotes hydrogen or alkyl having 1 to 7 carbon atom(s);
R³ is hydrogen;
R⁴ stands for hydrogen and R⁵ is a group of the general Formula (II), wherein R⁶, R⁷ and R⁸ each represents hydrogen, halogen, trifluoromethyl or straight or branched chain alkyl or alkoxy having 1 to 7 carbon atom(s), or R⁶ and R⁷ together form ethylenedioxy;
m is 1, 2, 3 or 4;
and pharmaceutically acceptable acid addition salts thereof.

3. 3-Alkyl indol-2-one derivatives of the general Formula (I),
wherein
R¹ stands for hydrogen, halogen or alkyl having 1 to 7 carbon atom(s);
R² represents hydrogen or alkyl having 1 to 7 carbon atom(s);
R³ denotes hydrogen;
R⁴ and R⁵ form, together with the adjacent carbon atoms of the tetrahydropyridine ring, phenyl or a 5- or 6-membered heterocyclic ring containing a sulfur as heteroatom, which may optionally carry a halogen substituent;
m is 1, 2, 3 or 4;
and pharmaceutically acceptable acid addition salts thereof.

4. 3-{4-[4-(3-Trifluoromethyl-phenyl)-1,2,3,6-tetrahydro-pyridin-1-yl]-butyl}-1,3-dihydro-2H-indol-2-one according to claim 1, and pharmaceutically acceptable acid addition salts there-of.

5. 3-[4-(6,7-Dihydro-4H-thieno[3,2-c]pyridin-5-yl)-butyl]-1,3-dihydro-2H-indol-2-one according to claim 1, and pharmaceutically acceptable acid addition salts thereof.

6. 3-[4-(6,7-Dihydro-4H-thieno[3,2-c]pyridin-5-yl)-butyl]-5-fluoro-1,3-dihydro-2H-indol-2-one according to claim 1, and pharmaceutically acceptable acid addition salts thereof.

7. 3-[4-(2-Chloro-6,7-dihydro-4H-thieno[3,2-c]pyridin-5-yl)-butyl]-1,3-dihydro-2H-indol-2-one according to claim 1, and pharmaceutically acceptable acid addition salts thereof.

8. 3-[4-(6,7-Dihydro-4H-thieno[3,2-c]pyridin-5-yl)-butyl]-6-fluoro-1,3-dihydro-2H-indol-2-one according to claim 1, and pharmaceutically acceptable acid addition salts thereof.

9. 3-[4-(2-Chloro-6,7-dihydro-4H thieno[3,2-c]pyridin-5-yl)-butyl]-6-fluoro-1,3-dihydro-2H-indol-2-one according to claim 1, and pharmaceutically acceptable acid addition salts thereof.

10. 3-[4-(2-Chloro-6,7-dihydro-4H-thieno[3,2-c]pyridin-5-yl)-butyl]-5-fluoro-1,3-dihydro-2H-indol-2-one according to claim 1, and pharmaceutically acceptable acid addition salts thereof.

11. 6-Fluoro-3-{4-[4-(3-trifluoromethyl-phenyl)-1,2,3,6-tetra-hydropyridin-1-yl]-butyl}-1,3-dihydro-2H indol-2-one monohydrochloride according to claim 1, and pharmaceutically acceptable acid addition salts thereof.

12. 3-{4-[4-(4-Chlorophenyl)-1,2,3,6-tetrahydro-pyridin-1-yl]-butyl}-1,3-dihydro-2H-indol-2-one according to claim 1, and pharmaceutically acceptable acid addition salts thereof.

13. 5-Fluoro-3-{4-[4-(3-trifluoromethyl-phenyl)-1,2,3,6-tetra-hydropyridin-1-yl]-butyl}-1,3-di-hydro-2H-indol-2-one according to claim 1, and pharmaceutically acceptable acid addition salts thereof.

14. Pharmaceutical compositions comprising as active ingredient at least one of the compounds of the general Formula (I) according to claim 1 or a pharmaceutically acceptable acid addition salt thereof in admixture with one or more conventional carrier(s) or auxiliary agent(s).

15. Pharmaceutical compositions according to claim 14 useful for the treatment or prophylaxis of central nervous disorders, particularly depression, anxiety, compulsory disorder, panic disease, social phobia, schizophrenia, mood disorders, mania, mental decline, stroke, cell death in certain parts of the central nervous system, neurodegeneration followed by mental decline, Alzheimer's disease, dementia, post-traumatic disease or stress disorder.

16. A process for the preparation of compounds of the general Formula (I), which comprises (a) reacting a compound of the general Formula (III), 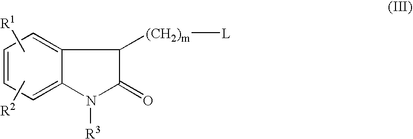
wherein L stands for hydroxy and R¹, R², R³ and m are as stated above, with an arylsulfonyl chloride or with a straight or branched chain C₁₋₇ alkylsulfonyl chloride in the presence of an organic base, and reacting the thus-obtained compound of the general Formula (III), wherein L represents aryl or alkylsulfonyloxy, with a pyridine derivative of the general Formula (IV), 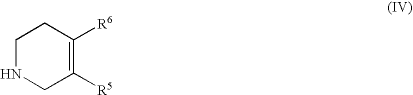
wherein R⁵ and R⁶ are as stated above, in the presence of an acid binding agent, or (b) reacting a compound of the general Formula (V), 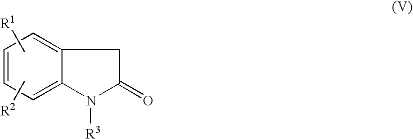
wherein R¹, R² and R³ are as stated above, with a compound of the general Formula (VII), 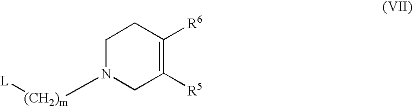
wherein R⁵, R⁶ and m are as stated above and L is a leaving group, in the presence of a strong base.

17. 3-Alkyl indol-2-one derivatives of the general Formula (I) according to claim 1 for use as a medicament.

18. A process for the manufacture of a pharmaceutical suitable for the treatment or prophylaxis of central nervous system disorders, particularly depression, anxiety, compulsive disorder, panic disease, social phobia, schizophrenia, mood disorders, mania, mental decline, stroke, cell death in certain areas of the central nervous system, mental decline followed by cerebellar cell death, Alzheimer's disease, dementia, post-traumatic disease or stress disorder, which comprises admixing at least one compound of the general Formula (I) according to claim 1 or a pharmaceutically acceptable acid addition salt thereof with a pharmaceutical carrier and optionally other auxiliary agent and bringing the mixture to galenic form.

19. A method for the treatment or prophylaxis of central nervous system disorders, particularly depression, anxiety, compulsive disorder, panic disease, social phobia, schizophrenia, mood disorders, mania, mental decline, stroke, cell death in certain areas of the central nervous system, mental decline followed by cerebellar cell death, Alzheimer's disease, dementia, posttraumatic disease or stress disorder, which comprises administering to a patient in need of such treatment an efficient amount of a pharmaceutical composition containing at least one compound of the general Formula (I) or a pharmaceutically acceptable, organic or inorganic acid addition salt thereof.
